(19) United States
(12) Patent Application Publication
(10) Pub. No.: US 2018/0049702 A1
(43) Pub. Date: Feb. 22, 2018

(54) SEMI-PENETRATING TYPE OPTICAL DETECTION APPARATUSES AND METHOD FOR PHYSIOLOGICAL CHARACTERISTIC(S)

(71) Applicant: PixArt Imaging Inc., Hsin-Chu City (TW)

(72) Inventor: Cheng-Nan Tsai, Hsin-Chu City (TW)

(21) Appl. No.: 15/390,726

(22) Filed: Dec. 27, 2016

(30) Foreign Application Priority Data
Aug. 16, 2016 (TW) 105126063

(51) Int. Cl. A61B 5/00; A61B 5/1455

(52) U.S. Cl. CPC A61B 5/7214; A61B 5/14552; A61B 2562/043; A61B 2562/0238; A61B 2562/0242; A61B 5/681

(57) ABSTRACT

An optical detection method for detecting physiological characteristic(s), includes: providing at least one light emitting unit; disposing the light emitting unit on a first plane to emit light to a skin surface position of a human body part to be detected of a user; providing at least one optical sensing circuit; disposing the optical sensing circuit on a second plane to receive a sensing result of Photoplethysmography (PPG) signal from another skin surface position of the human body part to be detected; and generating a physiological characteristic(s) detection result according to the sensing result; the first plane is different and distinct from the second plane, and a substantial angle is formed between the two plans.

### BACKGROUND OF THE INVENTION

### 1. Field of the Invention

The present invention relates to a physiological characteristic(s) detection scheme, and more particularly to a semi-penetrating type optical detection method and apparatus.

### 2. Description of the Prior Art

Generally speaking, a conventional optical detection scheme for physiological characteristic(s) is arranged to emit light rays at a certain light wavelength to a human body part of a user, receive a sensing result, and determine the physiological characteristic(s) of the user based on the absorption coefficient characteristic(s) of light ray energy for red blood cells of the human body part. The light rays at the certain light wavelength may be red light or infrared light.

However, actually, even adopting the red light or infrared light has been a better choice, the absorption coefficient characteristic(s) of red light or infrared light for the red blood cells of the human body part is still poor. This inevitably causes that an optical sensing circuit, used for receiving the sensing result, needs the higher signal-to-noise ratio (SNR) requirement for processing the sensing result. Unfortunately, the SNR of the sensing result is easily affected by a slight shaking of the human body part. For example, for measuring the degree of blood oxygen saturation (SpO2) of a wrist of a user, the SNR of a sensing result for the user's wrist is easily affected by the user's slight shaking such as the slight shaking of the wrist or human respiratory movement, and accordingly the measurement result is unstable.

### SUMMARY OF THE INVENTION

Therefore one of the objectives of the present invention is to provide a novel semi-penetrating type optical detection scheme for physiological characteristic(s), to detect a stronger signal, improve stability for the measurement, and to lower the SNR requirement for an optical sensing circuit, so as to solve the problems mentioned above.

According to embodiments of the present invention, an optical detection method for detecting physiological characteristic(s) is disclosed. The method comprises: providing at least one light emitting unit; disposing the light emitting unit on a first plane to emit light to a skin surface position on a human body part to be detected of a user; providing at least one optical sensing circuit; disposing the optical sensing circuit on a second plane to receive a sensing result of Photoplethysmography (PPG) signal from another skin surface position of the human body part to be detected; and generating a physiological characteristic(s) detection result according to the sensing result; wherein the first plane is different and distinct from the second plane, and a substantial angle is formed between the first plane and the second plane.

According to the embodiments, an optical detection apparatus for detecting physiological characteristic(s) is further disclosed. The optical detection apparatus comprises a main body and a belt body. The main body has a first portion and a second portion. The belt body is connected to or covers at least one portion of the main body, and is configured for fixing the main body to at least one human body part to be detected of a user. At least one light emitting unit is configured at the first portion, and is disposed on a first plane and used for emitting at least one light ray to at least one skin surface position of the at least one human body part to be detected of the user. At least one optical sensing circuit is configured at the second portion and disposed on a second plane and used for receiving a sensing result of Photoplethysmography (PPG) signal from another skin surface position of the human body part to be detected, to generate a physiological characteristic(s) detection result according to the sensing result. The first plane is different and distinct from the second plane, and a substantial angle is formed between the first plane and the second plane.

According to the embodiments, an optical detection apparatus for detecting physiological characteristic(s) is further disclosed. The optical detection apparatus comprises a main body and a belt body. The main body has a first portion. The belt body is connected to or covers at least one portion of the main body, and the belt body has a second portion and is configured for fixing the main body to at least one human body part to be detected of a user. At least one light emitting unit is configured at one of the first portion and the second portion, and is disposed on a first plane and used for emitting at least one light ray to at least one skin surface position of the at least one human body part to be detected of the user. At least one optical sensing circuit is configured at the other of the first portion and the second portion, disposed on a second plane, and used for receiving a sensing result of Photoplethysmography (PPG) signal from another skin surface position of the human body part to be detected, to generate a physiological characteristic(s) detection result according to the sensing result. The first plane is different and distinct from the second plane, and a substantial angle is formed between the first plane and the second plane.

According to the embodiments, an optical detection apparatus for detecting physiological characteristic(s) is further disclosed. The optical detection apparatus comprises a main body and a belt body. The belt body is connected to or covers at least one portion of the main body, and has a first portion and a second portion and is configured for fixing the main body to at least one human body part to be detected of a user. At least one light emitting unit is configured at one of the first portion and the second portion, and is disposed on a first plane and used for emitting at least one light ray to at least one skin surface position of the at least one human body part to be detected of the user. At least one optical sensing circuit is configured at the other of the first portion and the second portion, disposed on a second plane, and used for receiving a sensing result of Photoplethysmography (PPG) signal from another skin surface position of the human body part to be detected, to generate a physiological characteristic(s) detection result according to the sensing result. The first plane is different and distinct from the second plane, and a substantial angle is formed between the first plane and the second plane.

According to the embodiments, by appropriately adjusting relative positions, corresponding distance, and the angles of light projection/emission/sensing for optical sensing circuit(s) and light emitting unit(s), a semi-penetrating type optical detection scheme for the physiological characteristic(s) is provided and can be used to solve the problems of the conventional optical detection scheme.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a diagram showing a flowchart of an optical detection method for sensing/detecting physiological characteristic(s) according to an embodiment of the present invention.

FIG. 2A is a device diagram of a different modification embodiment according to the optical detection method as shown in FIG. 1.

FIG. 2B is a device diagram of another different modification embodiment according to the optical detection method as shown in FIG. 1.

FIG. 3A is a device diagram of another different modification embodiment according to the optical detection method as shown in FIG. 1.

FIG. 3B is a device diagram of another different modification embodiment according to the optical detection method as shown in FIG. 1.

FIG. 4A is a device diagram of another different modification embodiment according to the optical detection method as shown in FIG. 1.

FIG. 4B is a device diagram of another different modification embodiment according to the optical detection method as shown in FIG. 1.

FIG. 4C is a device diagram of another different modification embodiment according to the optical detection method as shown in FIG. 1.

FIG. 5A is a device diagram of another different modification embodiment according to the optical detection method as shown in FIG. 1.

FIG. 5B is a device diagram of another different modification embodiment according to the optical detection method as shown in FIG. 1.

FIG. 5C is a device diagram of another different modification embodiment according to the optical detection method as shown in FIG. 1.

FIG. 6 is a device diagram of another different modification embodiment according to the optical detection method as shown in FIG. 1.

### DETAILED DESCRIPTION

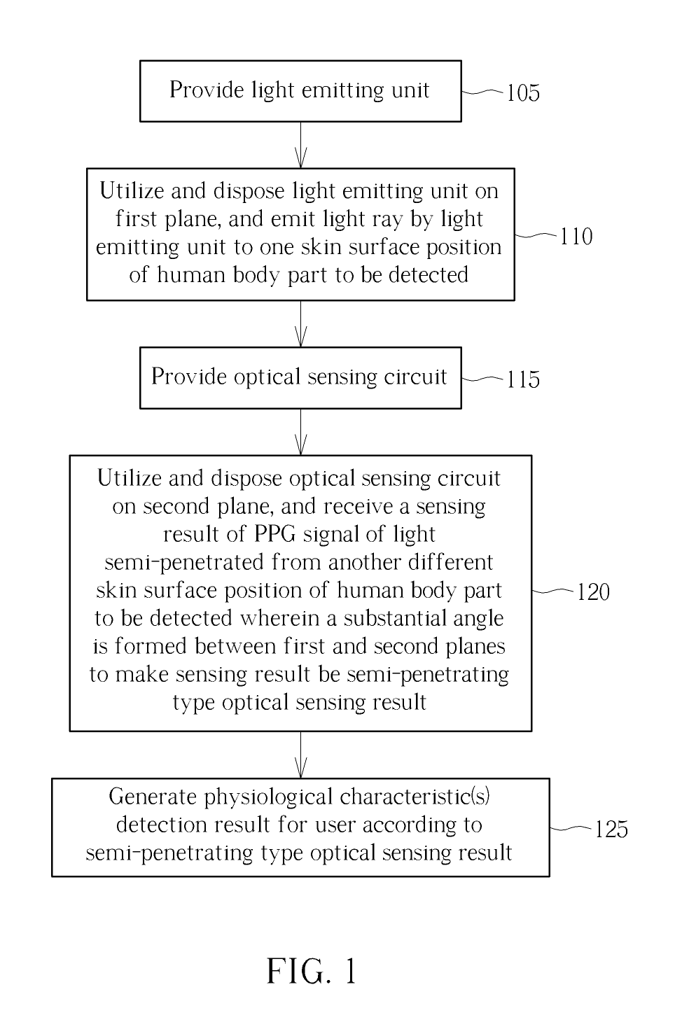

Refer to FIG. 1, which is a diagram showing a flowchart of an optical detection method for sensing/detecting physiological characteristic(s) according to an embodiment of the present invention. The optical detection method comprises the following steps:

Step 105: providing at least one light emitting unit such as a light emitting diode (LED) circuit;
Step 110: utilizing and disposing the light emitting unit on a first plane, and emitting at least one light ray by the light emitting unit to at least one skin surface position of at least one human body part to be detected of a user;
Step 115: providing at least one optical sensing circuit such as an LED sensing/receiving circuit;
Step 120: utilizing and disposing the optical sensing circuit on a second plane, and receiving a sensing result of a Photoplethysmography (PPG) signal of light semi-penetrated from another different skin surface position of the human body part to be detected of the user, the first plane is different and distinct form the second plane, and the two planes are positioned so that a substantial angle is formed between the two planes to make the sensing result be a semi-penetrating type optical sensing result; and
Step 125: generating a physiological characteristic(s) detection result for the user according to the semi-penetrating type optical sensing result.

As mentioned above, the optical detection method for detecting physiological characteristic(s) is used to detect a user's physiological characteristic(s) such as the heart pulse or arterial pulse through at least one skin surface position of at least one human body part to be detected of the user. In practice, the optical detection method employs a semi-penetrating type optical sensing/detecting scheme to detect the physiological characteristic(s) of the user, instead of using conventional schemes based on a sensing result of light reflection directly obtained from a skin surface position and/or a sensing result of light penetration directly penetrated through a human body part of the user. Compared to the above-mentioned conventional schemes, the optical detection method can improve the performance of optical detection/measurement for the physiological characteristic(s) significantly so that a stronger PPG signal can be detected/sensed. Accordingly, the optical detection method is capable of providing advantages of the stability improvement for the measurement of pulse oximetry (i.e., an estimate of the amount of oxygen saturation in the blood, SpO2) and reducing the signal-to-noise ratio (SNR) requirement for an optical sensing circuit. In addition, the optical detection method can be applied to a variety of electronic devices.

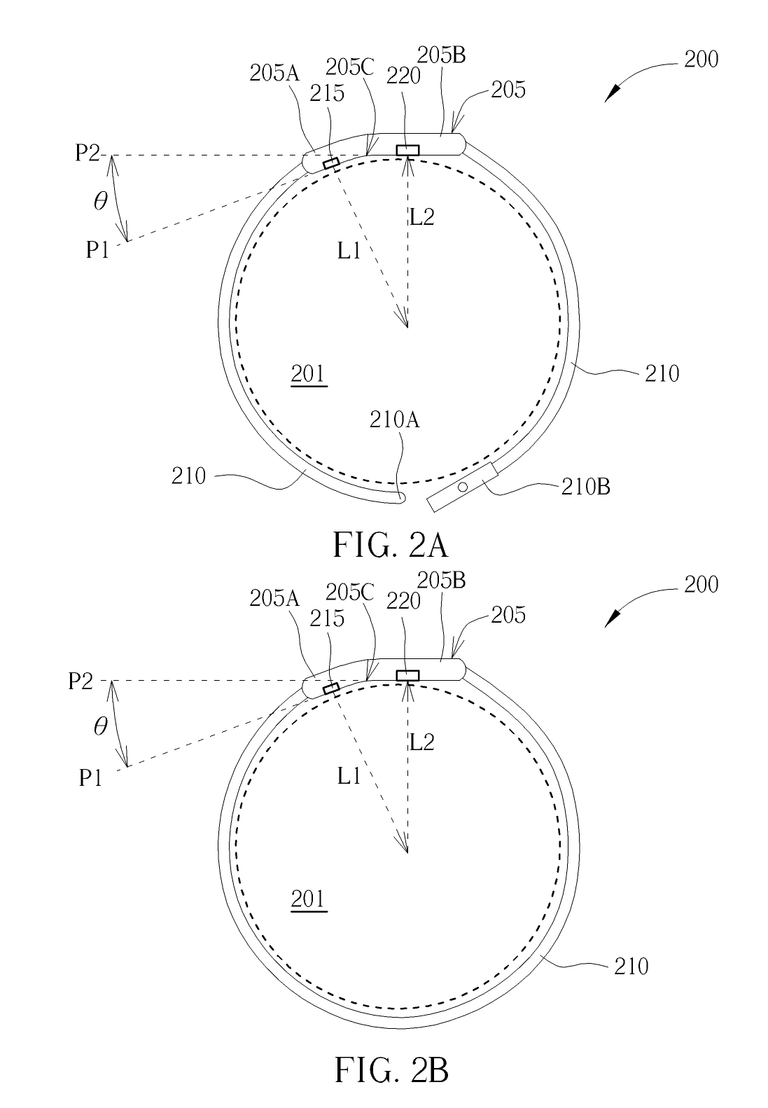

In an embodiment, the optical detection method for detecting physiological characteristic(s) can be applied to any kind of wearable electronic devices such as a wrist type wearable electronic device. Refer to FIG. 2A, which is a device diagram of an optical detection apparatus for detecting physiological characteristic(s) according to a first embodiment of the present invention. As shown in FIG. 2A, the optical detection apparatus 200 comprises a main body (main body device) 205 and a belt body 210 used as a fastening and holding element. The belt body 210 is connected to at least one portion of the main body 205 or cover the at least one portion, and includes two connection portions 210A and 210B at its two ends. The two connection portions 210A and 210B are utilized for fixing the main body 205 at the wrist 201 of a user wherein the wrist 201 is indicated by dotted curve. The optical detection apparatus 200 for example can be a smart watch device. The main body 205 can be connected to the belt body 210 via a pivot, a fastener, or other connection elements. The belt body 210 can be configured as different kinds of watch bands, watch straps, watch belts such as metal chain belts, leather belts, flexible watch bands, and/or expansion watch bands, and so on.

In addition, it should be noted that the optical detection apparatus 200 can be designed as the form of a bracelet. Refer to FIG. 2B, which is a diagram showing a modification embodiment of the optical detection apparatus for detecting physiological characteristic(s) as shown in FIG. 2A. As shown in FIG. 2B, if the optical detection apparatus 200 is implemented as a smart bracelet device, the belt body 210 can be designed as an integrally formed device having the same device housing. In this embodiment, the main body 205 and belt body 210 can be still connected via a pivot, a fastener, or other connection elements. It should be noted that the main body 205 and belt body 210 can be designed as a variety of modifications respectively. The above examples are not limitations of the present invention.

Refer back to FIG. 2A. The main body 205 includes a first portion 205A and a second portion 205B. The at least one light emitting unit 215 is configured at the first portion 205A, and the at least one optical sensing circuit 220 is configured at the second portion 205B. The first portion 205A and second portion 205B are connected via a concave part 205C to form a curved surface or a folded corner so that the first portion 205A and second portion 205B can be an integrally formed device and correspond to the same device housing. The concave part 205C can be designed as an inflexible concave part or a flexible concave part which can be adjusted by the user. By utilizing the concave part 205C, the at least one light emitting unit 215 at the first portion 205A and the at least one optical sensing circuit 220 at the second portion 205B can be disposed on two different and distinct planes P1 and P2, respectively. A substantial angle θ is formed between the two planes P1 and P2 and θ is between 0° and 180° (not equal to 0° or 180°). In another embodiment, the position of the at least one light emitting unit 215 and that of the at least one optical sensing circuit 220 can be exchanged. That is, the at least one light emitting unit 215 can be configured at the second portion 205B, and the at least one optical sensing circuit 220 can be configured at the first portion 205A. This modification also obeys the spirit of the invention.

For the light emitting unit 215, it includes an emission angular range of light ray emission wherein the emission angular range includes a dotted arrow L1 which is perpendicular to the first plane P1 and used for defining and indicating the central axis of the emission angular range for the emission of the light ray from the light emitting unit 205. The optical sensing circuit 220 includes a sensing/receiving angular range wherein the sensing/receiving angular range includes a dotted arrow L2 which is perpendicular to the second plane P2 and used for defining and indicating the central axis of the sensing/receiving angular range for the result of light ray semi-penetrated from a skin surface position of the human body part to be detected. Since the substantial angle θ is formed between the first and second planes P1 and P2, and accordingly the substantial angle θ is also formed between the central axis L1 of the emission angular range and central axis L2 of the sensing/receiving angular range.

When the optical detection apparatus 200 is wore or carried on the wrist 201 by the user, the light emitting unit 215 is arranged to emit light rays to a skin surface position of the wrist portion 201 of the user, and the optical sensing circuit 220 is arranged to sense and receive a result of absorption of energy at a certain light wavelength of the red blood cells from another different skin surface position of the wrist portion 201 to detect measurement of hemoglobin for the user. The two different skin surface positions are not located at opposite sides of the wrist portion 201, and the two different skin surface positions are not located at the same side of the wrist portion 201. The optical detection apparatus 200 is arranged to dispose the light emitting unit 215 and optical sensing circuit 220 at two different positions on two different planes P1 and P2 so that the light emitting unit 215 and optical sensing circuit 220 are not located oppositely to each other and can be separated to make a specified distance be left between the unit 215 and circuit 220. Accordingly, instead of sensing and receiving the measurement result obtained from light reflection at a skin surface position of the wrist portion or obtained from light penetration directly penetrated through the whole wrist portion, the optical sensing circuit 220 is capable of sensing and receiving a measurement result of absorption of energy at a certain light wavelength of the red blood cells, which is semi-penetrated through the human body part (i.e. the wrist portion 201) from one skin surface position to another different skin surface position. By doing so, the optical detection apparatus 200 is able to precisely detect measurement of hemoglobin for the user based on above-mentioned semi-penetrating type optical detection scheme. For example, in practice, the minimum distance left between the light emitting unit 215 and the optical sensing circuit 220 can be substantially designed as a distance between 1.5 cm and 8 cm; however, this is not intended to be a limitation.

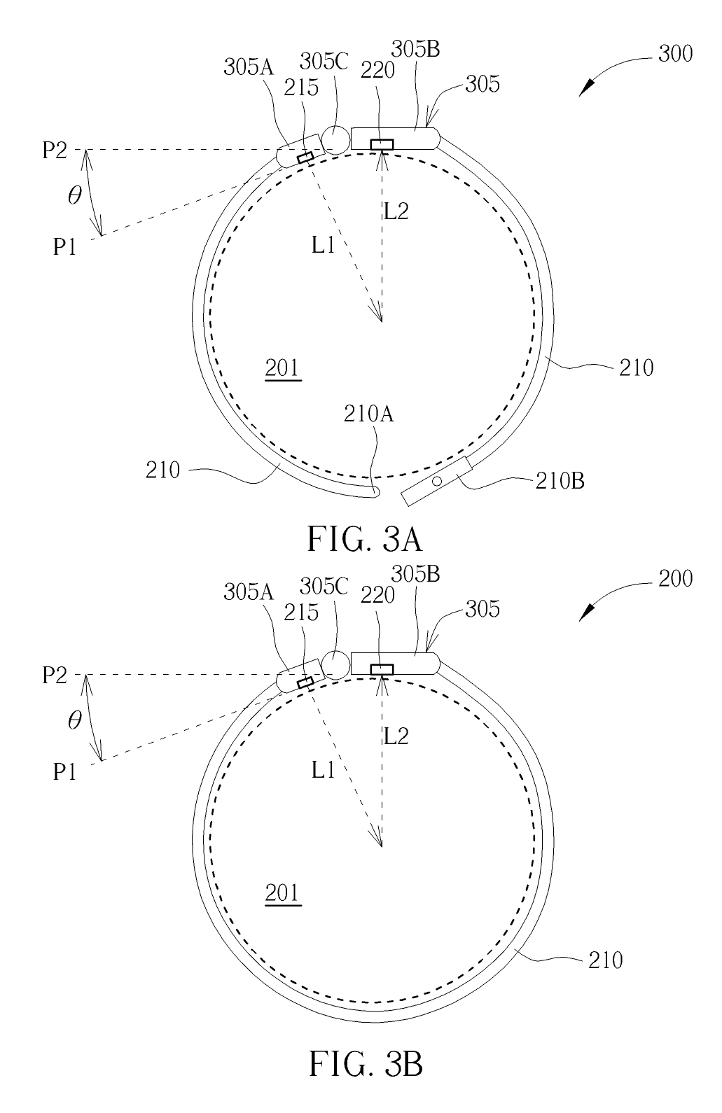

Furthermore, the optical detection method can be applied to different kinds of wrist type wearable electronic devices. Refer to FIG. 3A, which is a diagram showing an optical detection apparatus for detecting physiological characteristic(s) according to a second embodiment of the present invention. As shown in FIG. 3A, the optical detection apparatus 300 comprises a main body 305 and the belt body 210. The belt body 210 is connected to at least one portion of the main body 305 or covers the at least one portion, and includes two connection portions 210A and 210B at its two ends. The two connection portions 210A and 210B are utilized for fixing the main body 305 at the wrist portion 201 of the user. The optical detection apparatus 300 for example can be a smart watch device. The main body 305 can be connected to the belt body 210 via a pivot, a fastener, or other connection elements. When the belt body 210 convers the at least one portion of the main body 305 to fix the main body 305, the main body 305 and belt body 210 may be integrally formed.

In addition, it should be noted that the optical detection apparatus 300 can be designed as the form of a bracelet. FIG. 3B is a diagram showing another embodiment of the optical detection apparatus for detecting physiological characteristic(s) as shown in FIG. 3A. As shown in FIG. 3B, when the optical detection apparatus 300 is implemented as a smart bracelet device, the belt body 210 can be designed as an integrally formed device having the same device housing. In this embodiment, the main body 305 and belt body 210 can be still connected via a pivot, a fastener, or other connection elements. It should be noted that the main body 305 and belt body 210 can be designed as a variety of modifications respectively. The above examples are not limitations of the present invention.

Refer back to FIG. 3A. The main body 305 includes a first portion 305A and a second portion 305B. The at least one light emitting unit 215 is configured at the first portion 305A, and the at least one optical sensing circuit 220 is configured at the second portion 305B. The first portion 305A and second portion 305B are connected via a movable connection part 305C to form a curved surface or a folded corner. The movable connection part 305C can be designed as a pivot, a fastener or other connection elements to connect the first portion 305A and second portion 305B. For example, when the movable connection part 305C is implemented by a pivot, the pivot can be rotated so that different degrees of substantial angles can be formed between the first portion 305A and second portion 305B under different conditions. By using the movable connection part 305C, the light emitting unit 215 configured at the first portion 305A and the optical sensing circuit 220 configured at the second portion 305B can be respectively disposed on two different planes P1 and P2, and the substantial angle θ is formed between the two planes P1 and P2 and θ is between 0° and 90° (not equal to 0° or 90°). In another embodiment, the position of the at least one light emitting unit 215 and that of the at least one optical sensing circuit 220 can be exchanged. That is, the at least one light emitting unit 215 can be configured at the second portion 305B, and the at least one optical sensing circuit 220 can be configured at the first portion 305A. This modification also obeys the spirit of the invention.

Since the connection part 305C is movable, the first portion 305A and second portion 305B can be disposed in response to the shape of the wrist portion 201 of the user when the optical detection apparatus 300 is wore on the wrist portion 201 of the user. The light emitting unit 215 and optical sensing circuit 220 can be disposed on different planes P1 and P2 respectively. The dotted arrow L1 is perpendicular to the first plane P1 and used for defining and indicating the central axis of emission angular range for emission of light rays from the light emitting unit 205. The dotted arrow L2 is perpendicular to the second plane P2 and used for defining and indicating the central axis of sensing/receiving angular range for light sensing of light rays semi-penetrated from a skin surface position of the human body part (i.e. wrist portion 201) to be detected. The light emitting unit 215 is arranged to emit light ray to a skin surface position of the wrist portion 201 of the user, and the optical sensing circuit 220 is arranged to sense and receive a result of absorption of energy at a certain light wavelength of the red blood cells from another different skin surface position of the wrist portion 201 of the user to detect measurement of hemoglobin for the user. The two different skin surface positions are not located at opposite sides of the wrist portion 201, and are not located at the same side of the wrist portion 201. That is, the optical detection apparatus 300 is arranged to dispose the light emitting unit 215 and optical sensing circuit 220 at two different positions on two different planes so that the light emitting unit 215 and optical sensing circuit 220 are not located oppositely to each other and can be separated to make a specified distance be left between the unit 215 and circuit 220. Accordingly, instead of sensing and receiving the measurement result which is obtained from direct light reflection at a skin surface position of the wrist or light penetration directly penetrated through the whole wrist, the optical sensing circuit 220 is capable of sensing and receiving a measurement result of absorption of energy at a certain light wavelength of the red blood cells, semi-penetrated through the human body part (i.e. the wrist portion 201) from one skin surface position to another different skin surface position. By doing so, the optical detection apparatus 300 is able to precisely detect measurement of hemoglobin for the user based on above-mentioned semi-penetrating optical detection scheme.

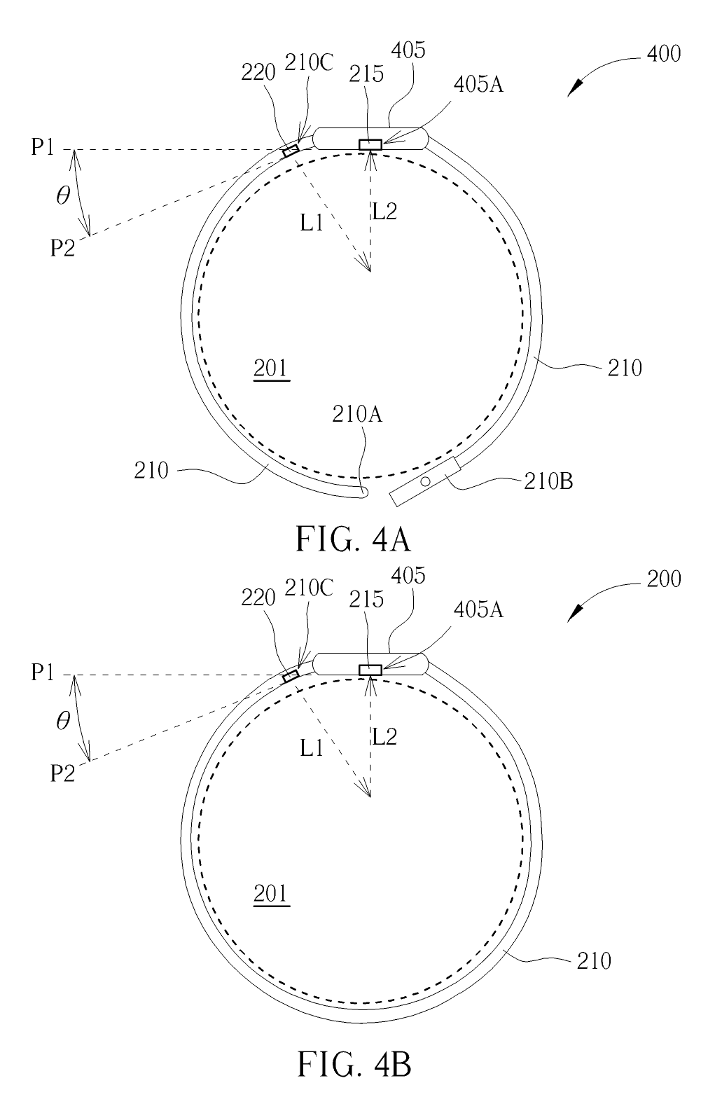

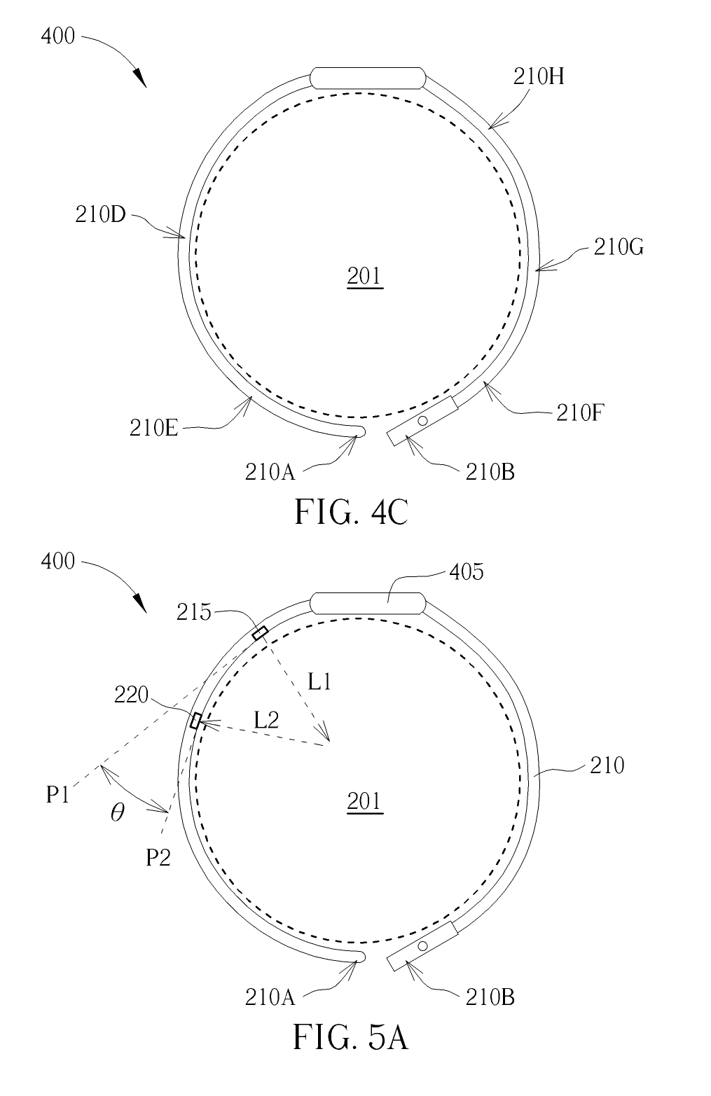

Furthermore, one of the light emitting unit 215 and optical sensing circuit 220 can be disposed/configured at a main body device, and the other is disposed/configured at a belt body. Refer to FIG. 4A, which is a diagram of an optical detection apparatus for detecting physiological characteristic(s) according to a third embodiment of the present invention. As shown in FIG. 4A, the optical detection apparatus 400 comprises a main body 405 and a belt body 210. The belt body 210 is connected to at least one portion of the main body 405 or covers the at least one portion, and includes two connection portions 210A and 210B at its two ends. The two connection portions 210A and 210B are utilized for fixing the main body 405 at the wrist 201 of a user. The optical detection apparatus 200 for example can be a smart watch device. The main body 405 can be connected to the belt body 210 via a pivot, a fastener, or other connection elements. The belt body 210 can be different kinds of watch bands, watch straps, watch belts such as metal chain belts, leather belts, flexible watch bands, and/or expansion watch bands.

In addition, it should be noted that the optical detection apparatus 400 can be designed as the form of a bracelet. Refer to FIG. 4B, which is a diagram showing a modification embodiment of the optical detection apparatus for detecting physiological characteristic(s) as shown in FIG. 4A. As shown in FIG. 4B, when the optical detection apparatus 400 is implemented as a smart bracelet device, the belt body 210 can be designed as an integrally formed device having the same device housing. In this embodiment, the main body 405 and belt body 210 can be still connected via a pivot, a fastener, or other connection elements. It should be noted that the main body 405 and belt body 210 can be designed as a variety of modifications respectively. The above examples are not limitations of the present invention.

The main body 405 includes a first portion 405A, and the belt body 210 further includes a second portion 210C, as shown in FIG. 4A. The at least one light emitting unit 215 is configured at the first portion 405A of the main body 405, and the at least one optical sensing circuit 220 is configured at the second portion 210C of the belt body 210. The first portion 405A and second portion 210C are connected via a pivot, a fastener, other connection elements, or an integrally informed flexible connection element between the main body 405 and belt body 210. The first portion 405A and second portion 210C can be disposed in response to the shape of the wrist portion 201 of a user when the optical detection apparatus 400 is wore on the wrist portion 201 of the user, so that the light emitting unit 215 and optical sensing circuit 220 can be disposed on two different planes P1 and P2 respectively wherein the substantial angle θ, having different degrees, can be formed between the planes P1 and P2. The operations and functions of the light emitting unit 215 and optical sensing circuit 220 have been described above and are not detailed for brevity. By configuring or disposing the at least one light emitting unit 215 at the main body 405 and configuring or disposing the at least one optical sensing circuit 220 at the belt body 210, the at least one light emitting unit 215 and the at least one optical sensing circuit 220 can be disposed at different positions on different planes. The light emitting unit 215 and optical sensing circuit 220 are not located at opposite sides of the wrist portion 201, and a specified distance is left between the unit 215 and circuit 220 to separate the unit 215 and circuit 220. Accordingly, instead of sensing and receiving the measurement result which is obtained from direct light reflection at a skin surface position of the wrist or light penetration directly penetrated through the whole wrist, the optical sensing circuit 220 is capable of sensing and receiving a measurement result of absorption of energy at a certain light wavelength of the red blood cells, semi-penetrated through the human body part (i.e. the wrist portion 201) from one skin surface position to another different skin surface position. By doing so, the optical detection apparatus 400 is able to precisely detect measurement of hemoglobin for the user based on above-mentioned semi-penetrating optical detection scheme.

In addition, the length of belt body 210 can be adjusted by the user when the user wears the optical detection apparatus 400 on his/her wrist portion. This function equivalently enables adjustments of the positions of light emitting unit 215 and optical sensing circuit 220 and corresponding distance left between the two elements. By user's adjustment, different degrees of substantial angles can be formed between the two planes P1 and P2, at which the light emitting unit 215 and optical sensing circuit 220 are configured. Because the user can adjust the length of belt body 210 by him or herself to accordingly adjust the skin surface position for optical sensing and receiving, the stability of measurement of SpO2 can be improved. it is more flexible for the user to use the optical detection apparatus 400 since the apparatus 400 can provide a variety of choices of skin surface positions for how to actually measure the physiological characteristic(s.

Furthermore, in other different embodiments, the optical sensing circuit 220 can be configured at different positions of the belt body 210. For example, as shown in FIG. 4C, the optical sensing circuit 220 can be configured at any positions 210D-210H. In addition, the positions of the light emitting unit 215 and optical sensing circuit 220 can be exchanged. That is, in another embodiment, the light emitting unit 215 can be configured at the belt body 210, and the optical sensing circuit 220 can be configured at the main body 405.

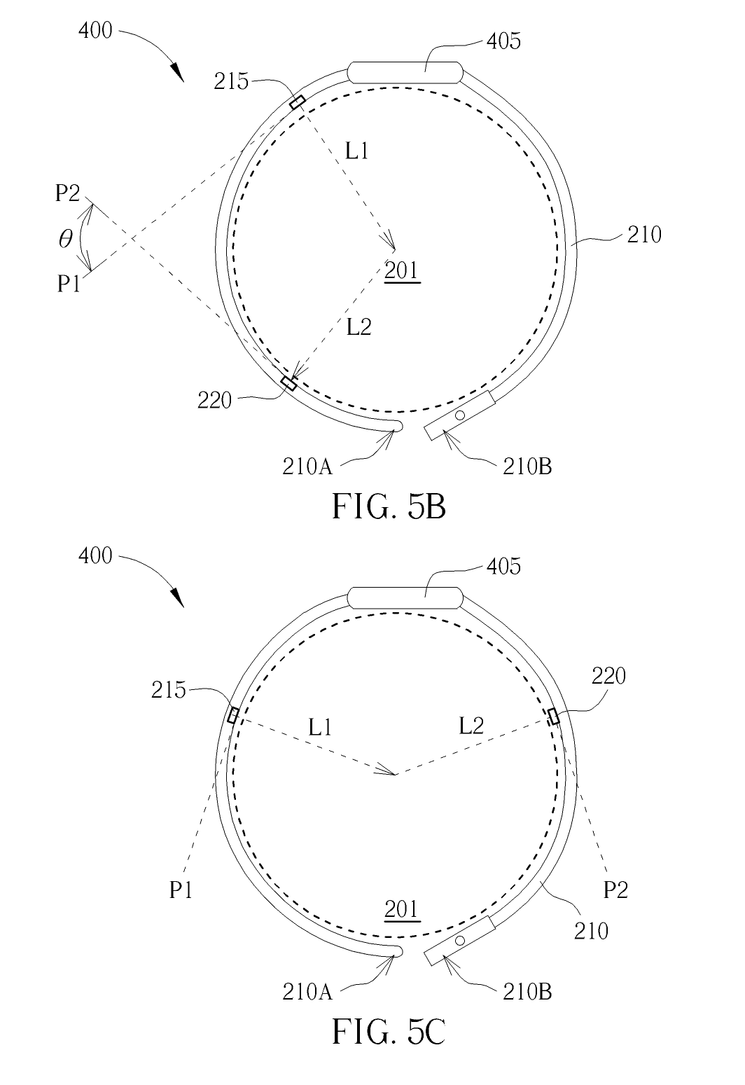

Additionally, in other embodiments, the light emitting unit 215 and optical sensing circuit 220 can be configured at different positions of the belt body 210, for example, as shown in FIG. 5A, FIG. 5B, and FIG. 5C respectively. The substantial angle θ is formed between the planes P1 and P2 and θ is between 0° and 180° (not equal to 0° or 180°). The belt body 210 is a flexible adjusting and bendable belt element, and the user can also adjust the length of the belt body 210 by him or her to correspondingly adjust the positions of the light emitting unit 215 and optical sensing circuit 220 to different positions on the planes respectively.

Additionally, in addition to apply the optical detection method to the above-mentioned wearable electronic devices, it is also appropriate to apply the optical detection method for detecting the physiological characteristic(s) by sensing and receiving a result of PPG signal obtained from a user's arm(s), finger(s), torso, or other human body parts. That is, the optical detection method and apparatuses are not limited by the wrist type wearable electronic device, and can be applied to an arm sleeve type electronic device, an armband type electronic device, a head mounted type electronic device, a headband type electronic device, and so on.

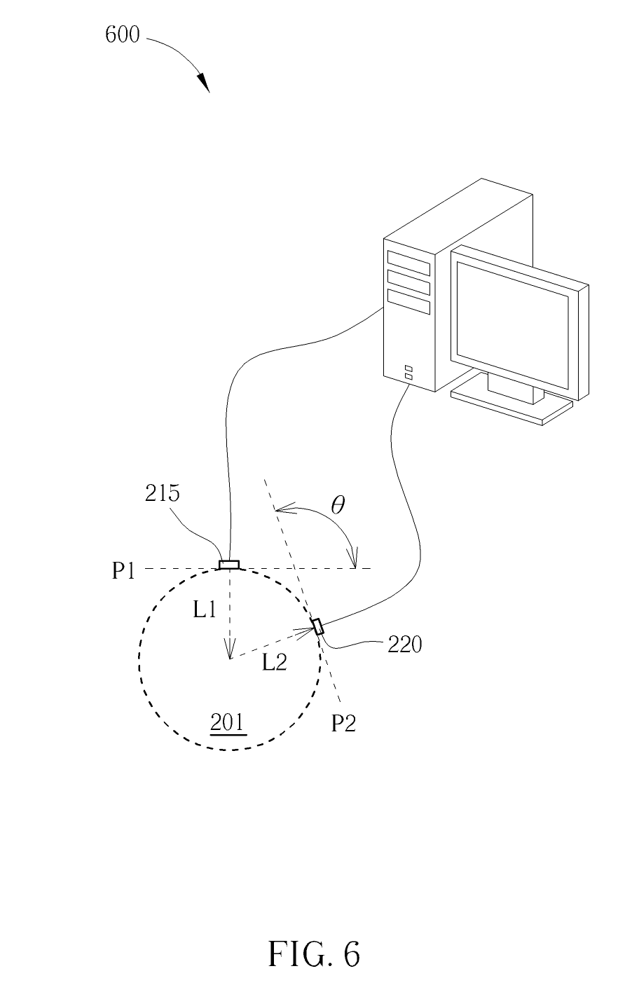

Additionally, the optical detection method can also be applied to a distributed computing system for detecting physiological characteristic(s). For example, as shown in FIG. 6, the light emitting unit 215 and optical sensing circuit 220 can be implemented by different and distinct hardware elements, respectively, and are connected to a computer system 600. The light emitting unit 215 and optical sensing circuit 220 are disposed on the different planes P1 and P2 (L1 and L2 are used for defining the central axis mentioned above), and are controlled by the computer system 600 to emit light ray to a skin surface position of a user and to receive a semi-penetrated sensing result obtained from a different skin surface position for detecting the physiological characteristic(s) of the user. This modification also falls within the scope of the invention.

To summarize, by appropriately adjusting relative positions, corresponding distance, and the angles of light projection/emission/sensing for optical sensing circuit(s) and light emitting unit(s) such as LED unit(s), the semi-penetrating type optical detection scheme for the physiological characteristic(s), provided by the embodiments of the invention, is able to solve the problems of the conventional detection scheme.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An optical detection method for detecting physiological characteristic(s), comprising:
providing at least one light emitting unit;
disposing the light emitting unit on a first plane to emit light to a skin surface position on a human body part to be detected of a user;
providing at least one optical sensing circuit;
disposing the optical sensing circuit on a second plane to receive a sensing result of Photoplethysmography (PPG) signal from another skin surface position of the human body part to be detected; and
generating a physiological characteristic(s) detection result according to the sensing result;
wherein the first plane is different and distinct from the second plane, and a substantial angle is formed between the first plane and the second plane.

2. The optical detection method of claim 1, wherein the substantial angle is between 0° and 180°.

3. An optical detection apparatus for detecting physiological characteristic(s), comprising:
a main body, having a first portion and a second portion; and
a belt body, connected to or covering at least one portion of the main body, configured for fixing the main body to at least one human body part to be detected of a user;
wherein at least one light emitting unit is configured at the first portion, the at least one light emitting unit is disposed on a first plane and used for emitting at least one light ray to at least one skin surface position of the at least one human body part to be detected of the user; at least one optical sensing circuit is configured at the second portion and disposed on a second plane and used for receiving a sensing result of Photoplethysmography (PPG) signal from another skin surface position of the human body part to be detected, to generate a physiological characteristic(s) detection result according to the sensing result; and, the first plane is different and distinct from the second plane, and a substantial angle is formed between the first plane and the second plane.

4. The optical detection apparatus of claim 3, wherein the substantial angle is between 0° and 180°.

5. The optical detection apparatus of claim 3, wherein the first portion and the second portion are integrally formed and are connected via a concave part to form a curved surface or a folded corner.

6. The optical detection apparatus of claim 3, wherein the first portion and the second portion are connected via a movable connection member to form a curved surface or a folded corner, and the movable connection member is a pivot or a fastener.

7. The optical detection apparatus of claim 3 is a wearable electronic device.

8. An optical detection apparatus for detecting physiological characteristic(s), comprising:
a main body, having a first portion; and
a belt body, connected to or covering at least one portion of the main body, the belt body having a second portion and being configured for fixing the main body to at least one human body part to be detected of a user;
wherein at least one light emitting unit is configured at one of the first portion and the second portion, the at least one light emitting unit is disposed on a first plane and used for emitting at least one light ray to at least one skin surface position of the at least one human body part to be detected of the user;
at least one optical sensing circuit is configured at the other of the first portion and the second portion, disposed on a second plane, and used for receiving a sensing result of Photoplethysmography (PPG) signal from another skin surface position of the human body part to be detected, to generate a physiological characteristic(s) detection result according to the sensing result; and, the first plane is different and distinct from the second plane, and a substantial angle is formed between the first plane and the second plane.

9. The optical detection apparatus of claim 8, wherein the substantial angle is between 0° and 180°.

10. The optical detection apparatus of claim 8 is a wearable electronic device.

11. An optical detection apparatus for detecting physiological characteristic(s), comprising:
a main body; and
a belt body, connected to or covering at least one portion of the main body, the belt body having a first portion and a second portion and being configured for fixing the main body to at least one human body part to be detected of a user;
wherein at least one light emitting unit is configured at one of the first portion and the second portion, the at least one light emitting unit is disposed on a first plane and used for emitting at least one light ray to at least one skin surface position of the at least one human body part to be detected of the user; at least one optical sensing circuit is configured at the other of the first portion and the second portion, disposed on a second plane, and used for receiving a sensing result of Photoplethysmography (PPG) signal from another skin surface position of the human body part to be detected, to generate a physiological characteristic(s) detection result according to the sensing result; and, the first plane is different and distinct from the second plane, and a substantial angle is formed between the first plane and the second plane.

12. The optical detection apparatus of claim 11, wherein the substantial angle is between 0° and 180°.

13. The optical detection apparatus of claim 11 is a wearable electronic device.
